(19) United States
(12) Patent Application Publication
(10) Pub. No.: US 2016/0250365 A1
(43) Pub. Date: Sep. 1, 2016

(54) An Unpowered Pressurized Chlorine Dioxide Automatic Generating and Mixing Disinfection Device

(71) Applicant: Shanghai Clean Bay Environmental Technology Co., Ltd., SHANGHAI (CN)

(72) Inventors: Zhimin MO, SHANGHAI (CN); Zhanning WANG, SHANGHAI (CN); Xiumin LI, SHANGHAI (CN)

(21) Appl. No.: 14/760,716

(22) PCT Filed: Mar. 18, 2015
(86) PCT No.: PCT/CN2015/074431

§ 371 Date: Jul. 14, 2015

(30) Foreign Application Priority Data
Aug. 25, 2014 (CN) 201410421943.1

(51) Int. Cl. A61L 2/18; C01B 11/02; B01J 14/00

(52) U.S. Cl. CPC A61L 2/18; B01J 14/00; C01B 11/024

(57) ABSTRACT

An unpowered pressurized chlorine dioxide automatic generating and mixing disinfection device having a first raw material tank, a second raw material tank, a reactor, a water injector and a check valve; the first and the second raw material tanks and the reactor are sealed container on which there are raw material inlets and discharge holes, one raw material inlet is connected to the first raw material tank by a first pick-up tube which connects with a first titration valve, the other is connected to the second raw material tank by a second pick-up tube which connects with a second titration valve; the reactor discharging hole is connected to the water injector inlet, the check valve is installed in the connecting pipe.

### BACKGROUND OF THE INVENTION

This inventions involves a device which generates chloride dioxide, mix with water and discharge automatically, it can be used as kitchen & toilet automatic disinfection device and is in the chlorine dioxide disinfection field.

Chlorine dioxide is a non-toxic oxidants and disinfectants, its disinfection capacity is efficient, broad-spectrum, no residue and no byproducts, and it has strong oxidizing ability to bleaching, deodorization and eradicating odor, it has strong ability to kill bacteria, viruses and fungal spores. Chlorine dioxide's disinfection and oxidation capacity are far better than chlorine, it will not generate harmful organic halides and trihalomethanes such carcinogenic substances, it can effectively destroy the phenol, sulfide, cyanide and other harmful substances, and it is the extremely effective disinfectant.

Normally chlorine dioxide is generated by filling hydrochloric acid and sodium chlorate (or sodium chlorite) quantitatively into the reaction tank, the reaction tank will have chemical reaction under heat and generate chlorine dioxide and chlorine, the chemical equation is:

NaClO₃+2HC1═NaC1+C1O₂1/2C1₂+H₂O;

5NaClO₂+4HCl═5NaCl+4ClO₂+2H₂O;

The sodium chlorate solution in certain concentration (or the sodium hypochlorite solution in certain concentration) and hydrochloric acid in certain concentration fill into the reaction tank quantitatively, the chlorine dioxide and chlorine gas-liquid mixture will be generated through aeration reaction under certain temperature and form the chlorine dioxide mixed disinfectant in certain concentration, then the mixture will be filled into water or objects to be disinfected with ejector, then the synergy disinfection and oxidation by chlorine dioxide and chlorine will be finished.

sodium chlorite (NaCLO₂) and citric acid (C₆H₈O₇) can also be used to react, the reaction equation is:

15NaCLO₂+4C₆H₈O₇═4Na₃C₆H₅O₇ (Sodium citrate)+12CLO₂+3NaCL+6H₂O.

Currently, the chlorine dioxide generator equipment is massive and expensive, and can not be operated under pressure. The quantitative transmission of sodium chlorate solution (or sodium chlorite solution) and hydrochloric acid is realized by metering system and electric control system, it will consume some power and need heat to react. For example, Chinese patent documents CN102701156A “An Chlorine Dioxide Generating System” includes heating reactor, the reactor both sides connect to the sodium chlorate and hydrochloric acid metering pump, the reactor top has a opening to discharge reaction product, the heating reactor bottom connects to the water injector which connects with system inflow circulation pump through pipe, the pipe is equipped with pressure gage, sodium chlorate metering pump connects with sodium chlorate tank, hydrochloric acid metering pump connects with hydrochloric acid tank, the manual valve connects sodium chlorate tank with mixing tank, the top of the mixing tank is connected with tap water pipe, the bottom of the mixing tank is connected with its circulation pump. This system not only consumes power, but only generates chlorine dioxide, it can not mix chlorine dioxide with water automatically.

CN102502507A publication “Chlorine Dioxide Generating Device and Process” includes reactor and chlorine dioxide cooler, the reactor is divided into at least three levels, including primary reactor, secondary reactor and three-level reactor; this device structure is complex. CN103334117A publication “A Chlorine Dioxide Generating Device with Efficient Electrolytic Method” consumes large energy.

CN103922288A publication “The Improved Structure of Chlorine Dioxide Generator” consists of storage tank and reactor; the storage tank is connected to sodium chlorate storage tank and hydrochloric acid storage tank by metering pump and reactor, the discharging holes of reactor is connected to processing water tank by water injector and discharging pipe, the entrance of water injector is equipped with power pipe. This structure only inject chlorine dioxide into processing water tank with water injector, it cannot meet immediate use (like tap water it can be mixed with water when tap is open, and stop automatically when close, it also can prevent tap water from backflowing into reactor), it can not satisfy kitchen and toilet automatic disinfection in home and hotels.

### BRIEF SUMMARY OF THE INVENTION

This invention aims to the disadvantages of existing chlorine dioxide disinfection technology, provides an unpowered pressurized automatic chlorine dioxide generating and mixing disinfection device, which structure is simple, doesn't need power and will not backflow into reactor even not used.

The unpowered pressurized automatic chlorine dioxide generating and mixing disinfection device of this invention employs following technology solutions:

This device mainly includes first raw material tank, second raw material tank, reactor, water injector and check valve; the reactor is a sealed container, which top has raw material inlets and discharge holes, one raw material inlet is connected to first raw material tank by first pick-up tube which connects to first titration valve, the other is connected to second raw material tank by second pick-up tube which connects to second titration valve; the reactor discharging hole is connected to the water injector inlet, the check valve shall be installed in the connecting pipe.

The said first raw material tank and second raw material tank all equipped with level indicator.

The said reactor has aeration hole which connects with aeration valve.

The said reactor bottom has slagging hole which is equipped with slagging valve to discharge reactor residue.

The said check valve consists of a housing and a valve plug, the valve plug is within the housing which one end has inlet and the other end is sealed, the housing side also has outlets.

When the above said device operates, the water injector inlet will be connected to the tap water pipe by inlet valve which will control the water inflow, the water injector outlet will connect to the drain pipe which will equip with drain valve. The different raw material required for reaction will be filled into two raw material tank. Open the inlet valve, titration valve and drain valve, then the tap water in certain pressure flows into the water injector and will be sprayed, meanwhile the suction chamber of water injector will have negative pressure so that the check valve is opened. Between tap water, water injector and drain pipe, it forms positive pressure water flow, but the reactor, first pick-up tube, second pick-up tube are under negative pressure operation. The liquid raw material in the raw material tank will be sucked into the reactor in certain flow and react to generate chlorine dioxide. The chlorine dioxide goes through the check valve into water injector and mixes with the high speed tap water, then it generates chlorine dioxide in certain concentration which becomes the disinfectant automatically. When outlet valve closes, the tap water stops flowing, then water injector has no negative pressure, tap water goes into check valve through water injector, the check valve closes, the negative pressure in the reactor disappears, the liquid raw material is not sucked into the reactor, and the reaction stops automatically.

This invention adds a check valve between water injector suction inlet (side hole) and reactor, then it can automatic transfer chlorine dioxide reaction raw material and generate chlorine dioxide without power automatically, meanwhile it can mix chlorine dioxide with water immediately and automatically to form the disinfectant; when tap water is closes, the reaction stops automatically so that tap water is prevented from backflowing into reactor. It is featured by simple structure, no power and convenient use.

### BRIEF DESCRIPTION OF THE DRAWINGS

FIG. 1 is the structure schematic diagram of this invention.

FIG. 2 is the structure schematic diagram of check valve involved in this invention.

FIG. 3 is the sectional schematic diagram of check valve plug.

Where: 1, inlet pipe; 2, inlet valve; 3, flow meter; 4, water injector; 5, check valve; 6, reactor; 7, aeration valve; 8, first titration valve; 9, second titration valve; 10, first pick-up tube; 11, second pick-up tube; 12, first raw material tank; 13, float level meter; 14, second raw material tank; 15, drain pipe; 16, drain valve; 17, delivery pipe; 18, suction inlet of water injector; 19, slagging hole; 20, inlet; 21, valve plug; 22, outlet; 23 end caps; 24, sealing ring; 25, housing.

### DETAILED DESCRIPTION OF THE INVENTION

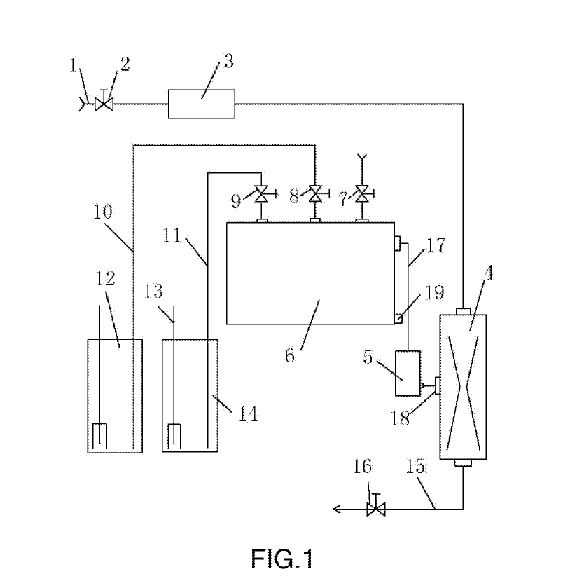

FIG. 1 shows the structure of this invention, the unpowered pressurized chlorine dioxide generating and mixing disinfection device, it mainly consists of first raw material tank 12, second raw material tank 14, reactor 6, water injector 4 and check valve 5. The first raw material tank 12 and the second tank 14 are filled sodium chlorite and citric acid aqueous solution respectively, the first raw material tank 12 is equipped with float level meter, and second raw material tank 14 is equipped with float level meter 13. Reactor 6 is a sealed container, which has two raw material inlets, one aeration hole and one drain outlet, raw material inlet and aeration hole are on the top and drain outlet is on the side. The first raw material inlet is connected to first raw material tank 12 by first pick-up tube 10, the first pick-up tube 10 is equipped with first titration valve 8, which can control liquid flow; The second raw material inlet is connected to second raw material tank 14 by second pick-up tube 11, the second pick-up tube 11 is equipped with second titration valve 9; the aeration hole is equipped with aeration valve 7; the outlet connects with inlet of water injector 4. Water injector 4 is a universal component, also known as ejector, it is made up by nozzle, suction chamber and diffuser tube three parts, correspondingly water injector 4 has inlets, suction inlets and outlets. Outlets of reactor 6 is connected to water injector suction inlet 18 by delivery pipe 17, which installs check valve 5, so that the chlorine dioxide generated by reactor 6 only can go to water injector 4, and the liquid in water injector 4 can not go to reactor 6.

When in specific application, the liquid inlet of water injector 4 connects with inlet tube 1, and inlet tube 1 connects to tap water pipe, inlet tube 1 has inlet valve 2 which can control water flow, inlet tube 1 also can connect with flow meter 3. The liquid outlet of water injector 4 connects with drain pipe 15 which has drain valve 16.

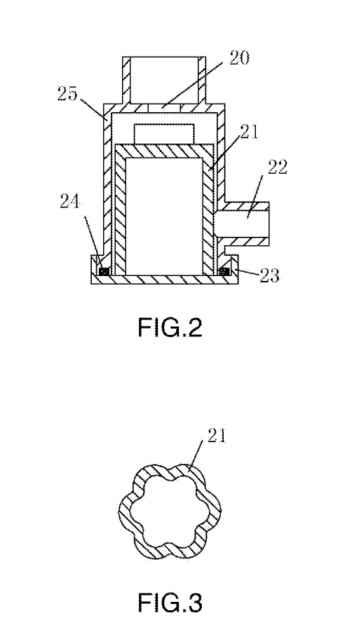

Check valve 5 can adopts existing common structure. In addition, check valve 5 can adopt the structure shown in FIG. 2 in order to control check valve 5 with little pressure, this structure consists of housing 25 and valve plug 21, valve plug 21 is within the housing 25, one end of housing 25 has inlet 20 the other end is sealed by end cap 23, side of housing 25 has outlet 22. Between end cap 23 and housing 25 side, there is a sealing ring 24. As a chamber with one end open and the other end sealed, the outer wall of valve plug 21 can have grooves, the cross section can be like quincunx as FIG. 3 shown. When the flowing medium (namely chlorine dioxides generated by reactor 6) flows into housing 25 through inlet 20, the valve plug 21 will be pushed down, then inlet 20 connects with outlet 22, the flowing medium discharges. When liquid (tap water) goes into housing 25 through outlet 22 under the injection of water injector 4, it flows into the bottom of valve plug 21 along the gap between valve plug 21 and housing 25, and makes the valve plug 21 float to plug the inlet 20, so that liquid is prevented from backflowing into reactor 6.

The bottom of reactor 6 can add a slagging hole 19 which fixes a slagging valve to discharge reaction residue regularly.

The operation of above said devices is as follows.

The raw material sodium chlorite solution and citric acid solution required by reaction will be filled into first raw material tank 12 and second raw material tank 14 respectively, the float level meter is used to watch level. Open inlet valve 2, first titration valve 8, second titration valve 9, aeration valve 7 and drain valve 16. The tap water under certain pressure comes into water injector 4 through inlet 1, and be sprayed out by water injector diffuser pipe, meanwhile the suction chamber of water injector has negative pressure, the air in reactor 6 has been exhaust by check valve 5, then reactor 6 inside is under negative pressure, so that check valve 5, reactor 6, first pick-up tube 10 and second pick-up tube 11 all operate under negative pressure; the liquid raw material in first raw material tank 12 and second raw material tank 14 have been sucked into reactor 6 in certain flow by first pick-up tube 10 and second pick-up tube 11 with the aid of first titration valve 8 and second titration valve 9 respectively. Sodium chlorite and citric acid (sodium chlorite and citric acid preferably for raw material) filled into reactor undergo chemical reaction and produce chlorine dioxide. The aeration valve 7 can control appropriate amount air to come into reactor 6, which will speed up the reaction. The chlorine dioxide goes into water injector 4 through reactor 6 drain outlet, delivery pipe 17 and check valve 5, and mixes with high-speed tap water, when the chlorine dioxide solution reaches certain concentration, it becomes the disinfectant automatically which can be used in kitchen to clean tableware, fruits and vegetables, and can be used in the toilet. The inlet valve 2 and flow meter 3 can control the concentration of chlorine dioxide solution.

When drain valve 16 is closed, tap water stops flowing out, the water injector 4 stops producing high-speed water flow, no negative pressure exists, the tap water comes into check valve 5 through suction inlet 18, it makes the valve plug 21 in check valve 5 float upward, then the inlet 20 of check valve 5 is closes, negative pressure in reactor 6 disappears, no liquid material is sucked into reactor 6, and the reaction stops automatically.

The above said devices make the disinfectant ready to use, the disinfectant water flows out when tap is open, and stops producing when tap is closed, no power is needed.

What is claimed is:

1. An unpowered pressurized chlorine dioxide automatic generating and mixing disinfection device comprising a first raw material tank, a second raw material tank, a reactor, a water injector and a check valve; wherein the reactor is a sealed container on which there are raw material inlets and discharge holes, one raw material inlet is connected to the first raw material tank by a first pick-up tube which connects with a first titration valve, the other is connected to the second raw material tank by a second pick-up tube which connects with a second titration valve; a reactor discharging hole is connected to an inlet of the water injector, the check valve is installed in a connecting pipe.

2. The device as in claim 1, wherein the first raw material tank and the second raw material tank all have level meter.

3. The device as in claim 1, wherein the reactor has aeration hole which connects with aeration valve.

4. The device as in claim 1, wherein the reactor bottome has slagging hole which are equipped with slagging valve.

5. The device as in claim 1, wherein the check valve consists of housing and valve plug, the valve plug is within the housing which one end has inlet and the other end is sealed, the housing side also has outlets.
